(19) United States
(12) Patent Application Publication
(10) Pub. No.: US 2008/0108149 A1
(43) Pub. Date: May 8, 2008

(54) Solid-phase mediated synthesis of molecular microarrays

(76) Inventors: Narayan Sundararajan, San Francisco, CA (US); John J. Rajasekaran, Los Altos, CA (US); Guangyu Xu, Milpitas, CA (US)

Correspondence Address: INTEL/BLAKELY, 1279 OAKMEAD PARKWAY, SUNNYVALE, CA, 94085-4040, US

(21) Appl. No.: 11/585,413

(22) Filed: Oct. 23, 2006

(51) Int. Cl. C12Q 1/68; A61K 38/00; G01N 33/543; C07K 1/00

(52) U.S. Cl. 436/518; 435/6; 530/344

(57) ABSTRACT

Methods for fabricating dense arrays of polymeric molecules in a highly multiplexed manner are provided using semiconductor-processing-derived methods and electrochemically generated reagents. Advantageously, the methods are adaptable to the synthesis of a variety of polymeric compounds. For example, arrays of peptides, polymers joined by peptide bonds, nucleic acids, and polymers joined by phosphodiester bonds may be fabricated in a highly multiplexed manner.

### CROSS REFERENCE TO RELATED APPLICATIONS

The present application is related to U.S. application Ser. No. 11/395,899, entitled “Massively Parallel Synthesis of Proteinaceous Biomolecules,” filed Mar. 30, 2006, now pending, U.S. application Ser. No. 11/144,679, entitled “Method and Apparatus to Fabricate Polymer Arrays on Patterned Wafers Using Electrochemical Synthesis,” filed Jun. 6, 2005, now pending, and U.S. application Ser. No. 11/207,000, entitled “Method and CMOS-based Device to Analyze Molecules and Nanomaterials Based on the Electrical Readout of Specific Binding Events on Functionalized Electrodes,” filed Aug. 19, 2005, now pending.

### BACKGROUND OF THE INVENTION

1. Field of the Invention

Embodiments of the invention relate generally semiconductor technology, to solid-phase synthesis of microarrays of bio-polymers using electrochemically generated reagents, and synthetic organic chemistry.

2. Background Information

Microarrays of oligonucleotides, peptides, proteins, and or oligosaccharides continue to gain importance as powerful tools for research and diagnostic applications in the biomedical sciences. For example, oligonucleotide microarrays can be used to monitor gene expression and genetic mutations and proteinaceous microarrays provide the ability to characterize the molecular progression of disease, research cellular pathways, and perform high throughput screening in drug discovery applications. Peptide-containing arrays can serve as molecular probes for a variety of biological events, such as for example, peptide arrays can serve as antigens for antibody-antigen systems, ligands for cell receptor-ligand system, and substrates for enzyme-protein systems. The ability to efficiently collect and analyze large volumes of information is an integral part of biomarker discovery and personalization of medical treatments. Further, other applications in bioscience, such as for example, the analysis of the proteomic content of an organism, disease detection, pathogen detection, environmental protection, food safety, and biodefense are capable of benefiting from tools that allow rapid multiplexed analysis of biomaterial samples.

As the genomic and proteomic knowledge base expands, so does the need for methods to collect, understand, and apply biologically relevant information. The drive towards personalized medicine magnifies these needs. Methods, such as analyses using microarrays that allow the use of small volumes of sample for highly multiplexed analysis, are valuable tools and accordingly, so are methods that provide for the controllable automated manufacture of arrays.

### BRIEF DESCRIPTION OF THE FIGURES

FIG. 1 diagrams a method for the controllable synthesis of polymers on a solid support using electrochemically generated reagents and semiconductor techniques.

FIG. 2 shows the step-by-step synthesis of polymers on a solid support.

FIGS. 3A, B, and C show chemical reactions that generate protons useful as catalysts for protective group removal.

FIG. 4 provides a method for derivatizing a SiO₂ surface and attaching a linker molecule to the derivatized surface.

FIG. 5 provides a diagram outlining a method for solid phase synthesis of a peptide.

FIG. 6 shows a CMOS switching scheme that provides individual addressability for electrodes on a surface.

### DETAILED DESCRIPTION OF THE INVENTION

Embodiments of the present invention provide methods for the synthesis and manufacture of polymer arrays. According to embodiments of the present invention, polymer arrays can be manufactured on a wafer scale in a massively parallel manner. High throughput synthesis of dense molecular arrays can be accomplished through the use of a solid phase catalytic or amplification layer and an array of electrodes. Electrochemical reactions generate a catalyst for protective group removal. A solid phase amplification layer that contains electro-active species is provided.

An array is an intentionally-created collection of molecules attached to a solid support in which the identity or source of a group of molecules is known based on its location on the array. The molecules housed on the array and within a feature of an array can be identical to or different from each other.

The features, regions, or sectors of an array may have any convenient shape, for example, circular, square, rectangular, elliptical, or wedge-shaped. In some embodiments, the region in which each distinct molecule is synthesized within a sector is smaller than about 1 mm², or less than 0.5 mm². In further embodiments the regions have an area less than about 10,000 μm², less than about 100 μm², or less than 2.5 μm². Additionally, multiple copies of a polymer will typically be located within any region. The number of copies of a polymer can be in the thousands to the millions within a region. In general, an array can have any number of features, and the number of features contained in an array may be selected to address such considerations as, for example, experimental objectives, information-gathering objectives, and cost effectiveness. An array could be, for example, a 20×20 matrix having 400 regions, 64×32 matrix having 2,048 regions, or a 640×320 array having 204,800 regions. Advantageously, the present invention is not limited to a particular size or configuration for the array. A plurality of arrays may be synthesized upon a silicon wafer and the wafer diced apart to provide separate arrays. Optionally, features of an array can be achieved by physically separating the regions into wells or trays.

A feature of an array could contain an electrode to generate an electrochemical reagent, a working electrode to synthesize a polymer, and a confinement electrode to confine the generated electrochemical reagent. The electrode to generate the electrochemical reagent could be of any shape, including, for example, circular, flat disk shaped and hemisphere shaped.

A monomer or a building block are molecules or compounds that can be joined together to form a polymer. The monomer or building block need not be limited to one monomeric unit and can be comprised of several units, that is, several monomeric units joined together. Monomers are joined by chemical bonds to form a polymer chain. The sequence of the polymer refers to the ordering of monomers in the polymer chain.

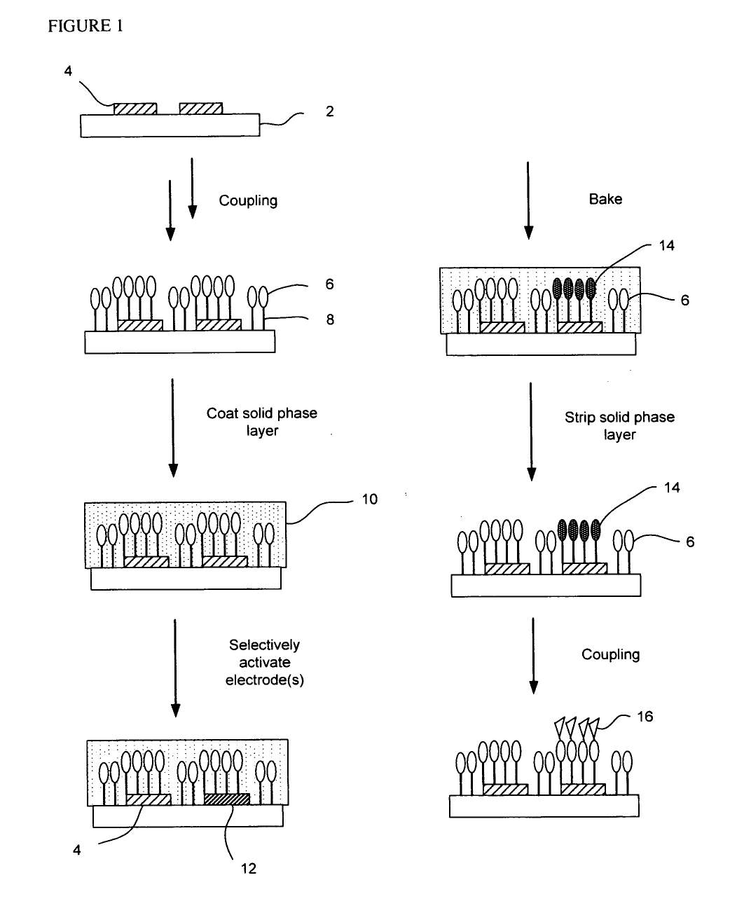

Referring now to FIG. 1, a method for synthesizing a polymer array on a solid substrate is provided. The substrate or silicon wafer 2 consists of an array of electrodes 4 that can be fabricated using semiconductor processing methods. A polymer building block having a protecting group 6 is attached to the solid substrate through a linker molecule 8 in a coupling reaction. As discussed more fully herein, in this example, the linker molecule serves to distance the polymer from the surface of the chip. In the case of peptide synthesis, the building block molecule 6 is an amino acid that is protected by, for example, a tert-butoxycarbonyl group. The surface is initially treated with oxygen plasma to generate an oxidized metal surface and the linker is coupled to the oxidized surface. Alternately, the surface may be coated with a thin porous SiO₂ layer and the linker attached through standard silane coupling chemistry. The surface is then coated with a thin solid-phase layer 10 that is capable of generating an acid (H⁺, protons) when exposed to a voltage of about −2 V to about +2 V, i.e., an amplification layer. The solid phase amplification layer is composed of matrix polymer (such as, for example, PMMA) dispersed with electro-sensitizers (molecules commonly used as redox pairs belonging to the quinine family such as hydroquinone, benzoquinone). Optionally, the solid phase layer can also contain amplifier molecules (termed electro-acid amplifiers (EAA)) that can amplify the generation of protons from protons generated from electro-sensitizers. The solid phase amplification layer serves to cleave protecting groups from the growing polymer chain in regions in which is activated by exposure to a voltage. Selected electrodes 12 are activated causing the proximate solid phase layer 10 to generate protons. The substrate is baked and the amplification layer is removed leaving two types of building blocks on the surface: the unmodified protected building block 6 and the deprotected building block 14. A second building block 16 is coupled to the deprotected first building block 14. This method can be repeated until the desired polymeric molecule(s) are synthesized on the substrate surface.

Similar approaches can be used for cleaving DMT (dimethoxytrityl) protecting groups for oligo nucleotide synthesis. Also, for base cleavable protecting groups such as F-moc groups, bases can be generated electrochemically along with base amplifiers (such as particular types of carbamates) in the solid phase layer for deprotection chemistry. This approach can also be used for small molecule synthesis (molecules having a molecular weight of less than about 800) generally done using principles currently applied in solution phase electrochemistry.

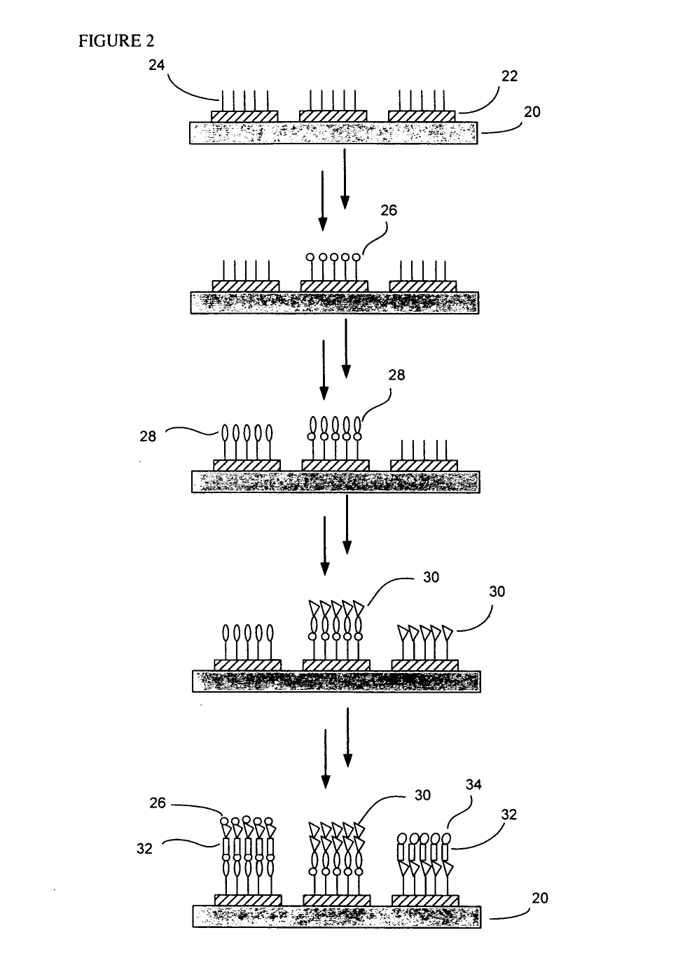

Referring now to FIG. 2, a general diagram showing the building of polymer molecules upon a substrate is provided. The substrate 20 contains an array of individually addressable electrodes 22. A protected spacer molecule 24 is coupled to the surface of the substrate 20. By selectively activating regions of the array, the protected molecule attached to the surface is prepared for coupling a second molecule through the removal of its protecting group. A protected polymer building block 26 is coupled to the deprotected surface-attached molecule. By repeatedly activating and deprotecting regions of the surface of the substrate building block molecules 26 through 34 are coupled to the surface of the substrate in a spatially specific manner.

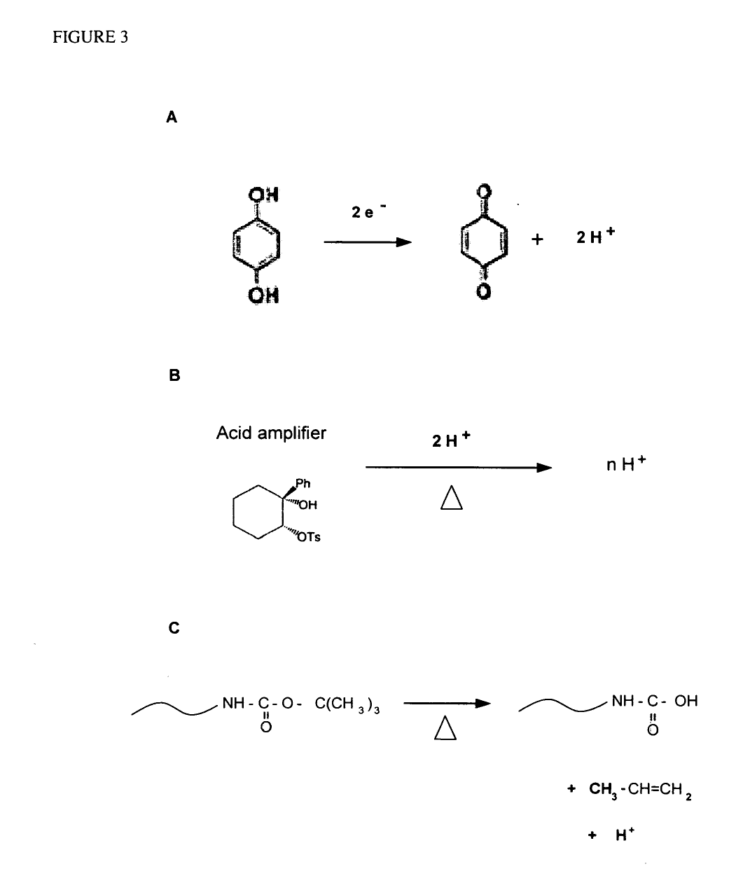

Electro-sensitizers (electroactive compounds) are compounds or molecules that can generate protons (H⁺) upon exposure to electrons. FIG. 3A provides an exemplary chemical reaction that may be used to generate protons in a solid-phase electroactive layer upon activation by an applied voltage. The electro-sensitizers that are dispersed in the solid phase amplification layer can be, for example, molecules commonly used as redox pairs belonging to the quinine family, such as, hydroquinone and benzoquinone.

Optionally, the amplification layer may also contain amplifier compounds that amplify the generation of protons from protons generated from electro-sensitizers (acid amplifier compounds). These amplifier molecules can be chosen from a class of molecules such as acid amplifiers (class of sulfonates undergoing autocatalytic fragmentation), photoacid generators such as, for example, onium salts such as diaryliodonium and triarylsulphonium salts, thermal acid generators, such as for example, 2,4,4,6-tetrabromocyclohexadienone, benzoin tosylate, 2-nitrobenzyl tosylate and other alkyl esters of organic sulfonic acids. FIG. 3B provides an exemplary chemical reaction for an amplifier compound. FIG. 3C provides an additional example of proton generation. In FIG. 3C, the heat-catalyzed removal of a t-butyl group produces propene and protons.

The electrodes that may be used in embodiments of the invention may be composed of, but are not limited to, metals such as iridium and/or platinum, and other metals, such as, palladium, gold, silver, copper, mercury, nickel, zinc, titanium, tungsten, aluminum, as well as alloys of these metals, and other conducting materials, such as, carbon, including glassy carbon, reticulated vitreous carbon, basal plane graphite, edge plane graphite, and graphite. Doped oxides such as indium tin oxide, and semiconductors such as silicon oxide and gallium arsenide are also contemplated. Additionally, the electrodes may be composed of conducting polymers, metal doped polymers, conducting ceramics and conducting clays.

The electrode(s) may be connected to an electric source in any known manner. Preferred ways of connecting the electrodes to the electric source include CMOS (complementary metal oxide semiconductor) switching circuitry, radio and microwave frequency addressable switches, light addressable switches, direct connection from an electrode to a bond pad on the perimeter of a semiconductor chip, and combinations thereof. CMOS switching circuitry involves the connection of each of the electrodes to a CMOS transistor switch. The switch could be accessed by sending an electronic address signal down a common bus to SRAM (static random access memory) circuitry associated with each electrode. When the switch is on, the electrode is connected to an electric source. Radio and microwave frequency addressable switches involve the electrodes being switched by a RF or microwave signal. This allows the switches to be thrown both with and/or without using switching logic. The switches can be tuned to receive a particular frequency or modulation frequency and switch without switching logic. Light addressable switches are switched by light. In this method, the electrodes can also be switched with and without switching logic. The light signal can be spatially localized to afford switching without switching logic. This could be accomplished, for example, by scanning a laser beam over the electrode array; the electrode being switched each time the laser illuminates it.

The generation of and electrochemical reagent of a desired type of chemical species requires that the electric potential of the electrode that generates the electrochemical reagent have a certain value, which may be achieved by specifying either the voltage or the current. The desired potential at an electrode may be achieved by specifying a desired voltage value or the current value such that it is sufficient to provide the desired voltage. The range between the minimum and maximum potential values is determined by the type of electrochemical reagent chosen to be generated.

In general, peptides are polymers of amino acids, amino acid mimics, amino acid derivatives, and/or unnatural amino acids which are generally joined together through amide (peptide) bonds. A peptide can alternatively be referred to as a polypeptide. Peptides contain two or more amino acid monomers, and often more than 50 amino acid monomers (building blocks). The amino acids can be any amino acids, including α, β, or ω-amino acids and modified amino acids. When the amino acids are α-amino acids, either the L-optical isomer or the D-optical isomer may be used. In general, an amino acid contains an amine group, a carboxylic group, and an R group. The R group can be a group found on a natural amino acid or a group that is similar in size to a natural amino acid R group. Additionally, unnatural amino acids, for example, β-alanine, phenylglycine, homoarginine, aminobutyric acid, aminohexanoic acid, aminoisobutyric acid, butylglycine, citrulline, cyclohexylalanine, diaminopropionic acid, hydroxyproline, norleucine, norvaline, ornithine, penicillamine, pyroglutamic acid, sarcosine, and thienylalanine are also contemplated by the embodiments of the invention. These and other natural and unnatural amino acids are available from, for example, EMD Biosciences, Inc., San Diego, Calif.

A protein is a long polymer of amino acids linked via peptide bonds and which may be composed of two or more polypeptide chains. More specifically, the term protein refers to a molecule comprised of one or more polymers of amino acids. Proteins are essential for the structure, function, and regulation of the body's cells, tissues, and organs, and each protein has unique functions. Examples of proteins include some hormones, enzymes, and antibodies.

Polynucleotide and oligonucleotide are used broadly herein to mean a sequence (polymer) of deoxyribonucleotides or ribonucleotides that are linked together by a phosphodiester bond. Generally, an oligonucleotide useful as a probe that selectively hybridizes to a selected nucleotide sequence is at least about 10 nucleotides in length, usually at least about 15 nucleotides in length, for example between about 15 and about 50 nucleotides in length. Polynucleotide probes are particularly useful for detecting complementary polynucleotides in a biological sample and can also be used for DNA sequencing. A polynucleotide can be a gene or a portion thereof, a cDNA, a synthetic polydeoxyribonucleic acid sequence, or the like. A polynucleotide, including an oligonucleotide (for example, a probe or a primer) can contain nucleoside or nucleotide analogs, or a backbone bond other than a phosphodiester bond. In general, the nucleotides comprising a polynucleotide polymer are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. One example of an oligomeric compound or an oligonucleotide mimetic that has been shown to have good hybridization properties is referred to as a peptide nucleic acid (PNA). In PNA compounds, the sugar-backbone of an oligonucleotide is replaced with an amide containing backbone, for example an aminoethylglycine backbone. In this example, the nucleobases are retained and bound directly or indirectly to an aza nitrogen atom of the amide portion of the backbone. PNA compounds are disclosed in Nielsen et al., Science, 254:1497-15 (1991), for example.

The covalent bond linking the nucleotides of a polynucleotide generally is a phosphodiester bond. However, the covalent bond also can be any of a number of other types of bonds, including a thiodiester bond, a phosphorothioate bond, a peptide-like amide bond or any other bond known to those in the art as useful for linking nucleotides to produce synthetic polynucleotides. The incorporation of non-naturally occurring nucleotide analogs or bonds linking the nucleotides or analogs can be particularly useful where the polynucleotide is to be exposed to an environment that can contain nucleolytic activity, including, for example, a tissue culture medium, since the modified polynucleotides can be less susceptible to degradation.

A linker molecule typically is a molecule inserted into the growing polymer that does not necessarily convey functionality to the resulting polymer, such as molecular recognition functionality, but instead elongates the distance between the substrate surface and the polymer functionality to enhance the exposure of the peptide functionality on the surface of the substrate. Typically a linker molecule is about 4 to about 40 atoms long. The linker molecules may be, for example, aryl acetylene, ethylene glycol oligomers containing 2-10 monomer units (PEGs), diamines, diacids, amino acids, among others, and combinations thereof. Examples of diamines include ethylene diamine and diamino propane. Alternatively, the linkers may be the same molecule type as that being synthesized (i.e., nascent polymers), such as, polynucleotides, peptides, oligosaccharides, or polymers of amino acid derivatives such as for example, amino hexanoic acids.

A protecting group (or protective group) is a chemical functional group that is bound to a molecule and designed to block a reactive site of the molecule. A protecting group may be removed upon exposure to an activator or a deprotecting reagent. The selection of protecting group for a particular synthesis is governed by the overall methods employed in the synthesis. Activators include, for example, electromagnetic radiation, ion beams, electric fields, magnetic fields, electron beams, x-ray, and the like. A deprotecting reagent could include, for example, an acid, a base, or a free radical.

Additional protecting groups that may be used in accordance with embodiments of the invention include acid labile groups for protecting amino moieties: tert-amyloxycarbonyl, adamantyloxycarbonyl, 1-methylcyclobutyloxycarbonyl, 2-(p-biphenyl)propyl(2)oxycarbonyl, 2-(p-phenylazophenylyl)propyl(2)oxycarbonyl, .alpha.,.alpha.-dimethyl-3,5-dimethyloxybenzyloxy-carbonyl, 2-phenylpropyl(2)oxycarbonyl, 4-methyloxybenzyloxycarbonyl, furfuryloxycarbonyl, triphenylmethyl (trityl), p-toluenesulfenylaminocarbonyl, dimethylphosphinothioyl, diphenylphosphinothioyl, 2-benzoyl-1-methylvinyl, o-nitrophenylsulfenyl, and 1-naphthylidene; as base labile groups for protecting amino moieties: 9-fluorenylmethyloxycarbonyl, methylsulfonylethyloxycarbonyl, and 5-benzisoazolylmethyleneoxycarbonyl; as groups for protecting amino moieties that are labile when reduced: dithiasuccinoyl, p-toluene sulfonyl, and piperidino-oxycarbonyl; as groups for protecting amino moieties that are labile when oxidized: (ethylthio)carbonyl; as groups for protecting amino moieties that are labile to miscellaneous reagents, the appropriate agent is listed in parenthesis after the group: phthaloyl (hydrazine), trifluoroacetyl (piperidine), and chloroacetyl (2-aminothiophenol); acid labile groups for protecting carboxylic acids: tert-butyl ester; acid labile groups for protecting hydroxyl groups: dimethyltrityl. See also, Greene, T. W., Protective Groups in Organic Synthesis, Wiley-Interscience, NY, (1981).

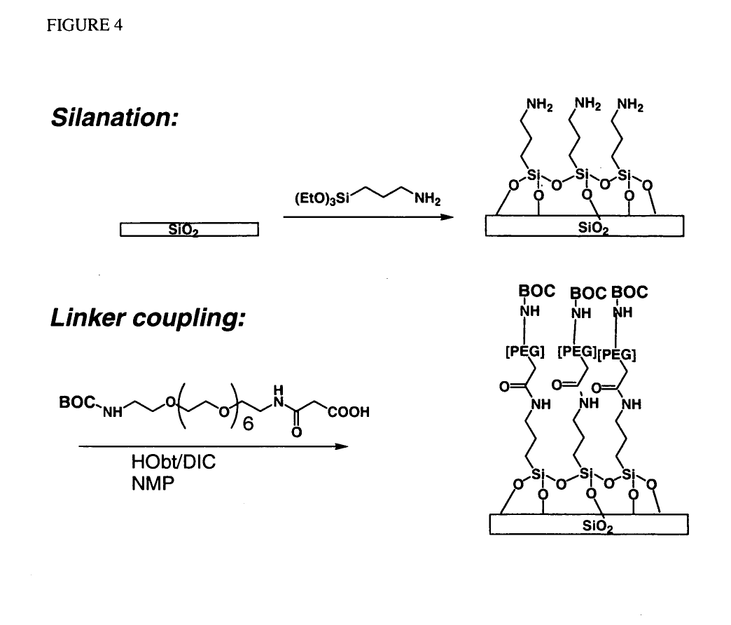

FIG. 4 provides a method for derivatization of a SiO₂ surface and linking of polymeric molecules to the surface. In FIG. 4 the SiO₂ surface is silanated by reacting it with aminopropyltriethoxy silane (APTES). The resulting surface presents an amine functional group for further reaction, such as peptide bond formation. Modulation of the density of polymers on the surface can be attained by silanation. For example, density can be modulated by mixing a functionalizable silane for example, APTES, with a non-functional silane (a silane with no non silyl functional group), for example, propyltrialkoxy silane. The derivatized surface can then be reacted with a linker. In this example, the linker is a polyethylene glycol molecule having an amine group protected with BOC at one terminus and a peptide-bond forming group at the second terminus. This coupling reaction can be accomplished in a solution of 1-hydroxybenzotriazole (HOBt) and diisopropylcarbodiimide (DIC) in N-methylpyrrolidone (NMP). The linker molecule serves to separate polymer (peptide) that is subsequently synthesized from surface of the substrate.

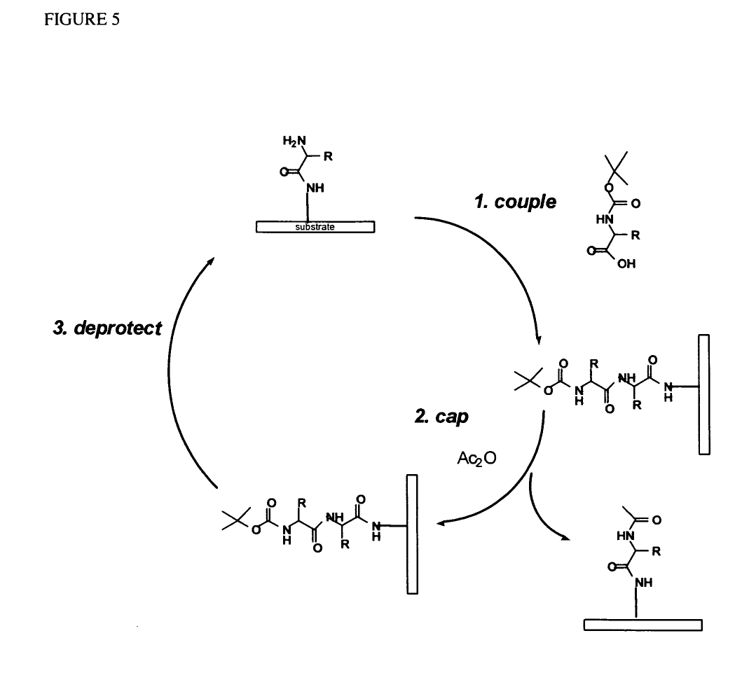

FIG. 5 shows a general scheme for solid-phase peptide synthesis. A substrate surface is provided having a first amino acid attached to the surface. A second amino acid having a protecting group is coupled to the first amino acid. In this example, the second amino acid is N-protected with a BOC protecting group. The coupling reaction is performed in a solution of 1-hydroxybenzotriazole (HOBt) and diisopropylcarbodiimide (DIC) in N-methylpyrrolidone (NMP). Unreacted amine groups are capped using an acetic anhydride (Ac₂O) solution in dimethylformamide (DMF). The substrate surface is then coated with a solid phase amplification layer. Upon activation of an electrode in a region of the substrate, an acid is produced in the solid phase layer adjacent to the electrode and the N-protecting group is removed from the attached peptide. By repeating the process shown in FIG. 5, peptides of desired sequence and length is selected regions upon the substrate surface can be produced.

The solid phase coating in embodiments of the invention consists, in part of a polymer. Useful polymers include, for example, poly(methyl methacrylate) (PMMA), poly-(methyl isopropenyl ketone) (PMPIK), poly-(butene-1-sulfone) (PBS), poly-(trifluoroethyl chloroacrylate) (TFECA), copolymer-(α-cyano ethyl acrylate-α-amido ethyl acrylate (COP), and poly-(2-methyl pentene-1-sulfone). Useful solvents include, for example, propylene glycol methyl ether acetate (PGMEA), ethyl lactate, and ethoxyethyl acetate. The solvent used in fabricating the solid phase layer may be selected depending on the particular polymer, electroactive species, and amplification species that are selected.

In exemplary solid phase coating formulations, the mass concentration of the polymer may between about 5% and about 50%, the mass concentration of a electroactive species may be up to about 20%, the mass concentration of the optional acid amplifier may be between about 1% and 10%, the balance comprising a suitable solvent. After the solid phase polymer solution is deposited on the substrate, the substrate typically is heated to form the solid phase layer. Any method known in the art of semiconductor fabrication may be used to for depositing the solid phase layer solution. For example, the spin coating method may be used in which the substrate is spun typically at speeds between about 1,000 and about 5,000 revolutions per minute for about 30 to about 60 seconds. The resulting wet solid phase layer has a thickness ranging between about 0.1 μm to about 2.5 μm.

Solid support, support, and substrate refer to a material or group of materials having a rigid or semi-rigid surface or surfaces. In some aspects, at least one surface of the solid support will be substantially flat, although in some aspects it may be desirable to physically separate synthesis regions for different molecules with, for example, wells, raised regions, pins, etched trenches, or the like. In certain embodiments, the solid support may be porous.

A wafer is a semiconductor substrate. A wafer could be fashioned into various sizes and shapes. It could be used as a substrate for a microchip. The substrate could be overlaid or embedded with circuitry, for example, a pad, via, an interconnect or a scribe line. The circuitry of the wafer could also serve several purposes, for example, as microprocessors, memory storage, and/or communication capabilities. The circuitry can be controlled by the microprocessor on the wafer itself or controlled by a device external to the wafer.

A via interconnection refers to a hole etched in the interlayer of a dielectric which is then filled with an electrically conductive material, for example, tungsten, to provide vertical electrical connection between stacked up interconnect metal lines that are capable of conducting electricity. A scribe line is typically an inactive area between the active dies that provide area for separating the die. Often metrology and alignment features populate this area.

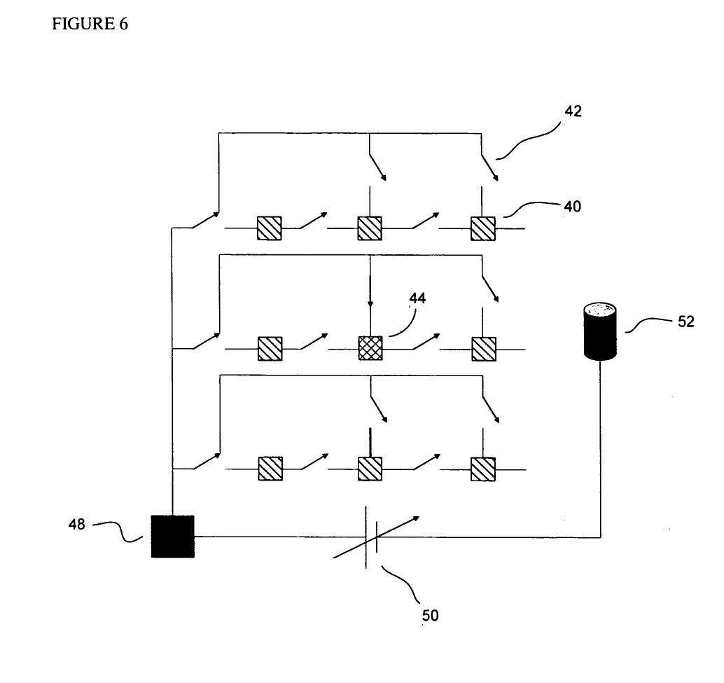

Array chips on silicon wafers can be built using silicon process technology and SRAM like architecture with circuitries including electrode arrays, decoders, and serial-peripheral interface, for example. Individually addressable electrodes can be created with CMOS circuitry. The CMOS circuitry, among other functions, amplifies the signal, and reads and writes information on the individually addressable electrodes. FIG. 6 shows a CMOS switching scheme for individually addressing different working electrodes on a wafer. In FIG. 6, each die pad on the die branches into a large array of synthesis electrodes. CMOS switches ensure that a given electrode (or an entire column, or an entire row) can be modified one base pair at a time. Voltage source and counter electrode (plating tool) are shown to complete the electrical circuit. In FIG. 6, the electrodes of the array 40 are electrically connected through a CMOS switch 42 through a bonding pad 48 to a voltage source 50. A counter electrode 52 is also supplied. With this scheme, and electrode 44 can be individually activated. The bonding pad 52 is used, for example, for power and signal delivery. The die pads could be interconnected by either using a multilevel interconnect (two or more layers) across a scribe line on the front side of the wafer or by using a via interconnect that traverses from the front side of the wafer to the backside of the wafer.

### EXAMPLE

The substrate is a silicon wafer that consists of an array of individually-addressable electrodes that is fabricated using traditional semiconductor processing methods. A thin porous SiO₂ layer is applied to the surface of the electrode. The surface is functionalized with 0.5% aminopropyl triethoxy silane (APTES) in ethanol for 30 minutes, washed with ethanol, and subsequently cured at 110° C. for 1 hour. A spacer molecule is then coupled to the surface using a 0.25 M solution of O-(N-Boc-2-aminoethyl)-O′-(N-diglycolyl-2-aminoethyl)hexaethyleneglycol, 0.25 M HOBt, and 0.25 M DIC (diisopropylcarbodiimide) in NMP (N-methylpyrrolidone) for about 30 min. After coupling is complete, the surface is washed with NMP and then acetone. Unreacted surface amine groups are capped by treatment with 1:1 acetic anhydride in DMF solution (a 50% acetic anhydride solution in DMF) for about 30 minutes. T-BOC protected glycine is coupled to the amino functionalized surface at 0.1 M concentration in a solution containing 0.1 M DIC and HOBt (diisopropyl carbodiimide and hydroxybenzotriazole, activators) in N-methyl-2-pyrrolidinone (NMP) for 30 min. The unreacted amino groups of the surface are capped using a 50% acetic anhydride solution in dimethylformamide (DMF) for 30 min. A thin film (about 100 nm to about 500 nm thick) of solid phase amplification layer is spin coated at 2000 rpm for 60 sec in a spin coater on top of the substrate and the substrate is baked at 85° C. for 90 sec. The solid phase amplification layer is composed of matrix polymer, PMMA, having a dispersed electro-sensitizer, hydroquinone. The solid phase layer also contains an amplifier, benzoin tosylate, that can amplify the generation of protons from protons generated from electro-sensitizers. Electrodes are selectively activated and provide a voltage to regions that are to have an amino acid coupled to the building block molecule on the surface of the substrate in order to remove the t-BOC group. The substrate is then baked at 110° C. for 1 hour and the solid phase layer is removed. The surface of the substrate is treated with a solution of t-BOC-leu-OH, 1-hydroxybenzotriazole (HOBt), and diisopropylcarbodiimide (DIC) in N-methyl pyrrolidone (NMP). The unreacted amino groups of the surface are capped using a 50% acetic anhydride solution in dimethylformamide (DMF) for 30 min. The regions that contain the selectively activated electrodes contain a two amino acid sequence of glycine-leucine.

We claim:

1. A method for polymer synthesis on a solid support comprising,
providing an array of electrodes on a surface of the solid support;
attaching a first molecule capable of forming a peptide bond wherein the molecule contains a protecting group that prevents the formation of a peptide bond to the support surface;
coating a solid phase polymer layer on the surface of the solid support, wherein the solid phase polymer layer contains an electroactive compound that upon activation generates a second compound capable of catalyzing the removal of the protecting group;
activating the electroactive compound on at least a portion of the surface of the solid support by supplying a voltage to at least one electrode of the array of electrodes;
removing the solid phase polymer layer; and
coupling a second molecule capable of forming a peptide bond, wherein the molecule contains a protecting group that prevents the formation of a peptide bond, to a first molecule capable of forming a peptide bond that has been deprotected.

2. The method according to claim 1 also including heating the solid support after supplying a voltage to at least one electrode of the array of electrodes.

3. The method according to claim 1 also including capping any unreacted deprotected peptide bond-forming sites on the first molecule capable of forming a peptide bond after coupling the second molecule capable of forming a peptide bond.

4. The method according to claim 1 wherein attaching is accomplished through the formation of a peptide bond.

5. The method of claim 1 wherein the solid phase polymer layer also contains an acid amplifier compound.

6. The method of claim 1 wherein the protecting group is selected from the group consisting of t-butoxycarbonyl, benzyloxycarbonyl, and 9-fluorenylmethoxycarbonyl.

7. The method of claim 1 wherein a feature size of the array is less than 100 μm².

8. The method of claim 1 wherein the array contains 1,000 to 10,000 electrodes.

9. The method of claim 1 wherein the method is repeated to form at least one polymer having at least 7 peptide bonds.

10. The method of claim 1 wherein the solid support is a silicon wafer.

11. The method of claim 1 wherein the solid support contains at least 500 electrodes that are individually addressable.

12. A method for making an array of polymers comprising on a solid support comprising,
modulating the density of polymers to be formed on a surface of the solid support by blocking a fraction of the possible attachment sites on the support surface from molecular coupling;
attaching to the surface a first molecule capable of forming a peptide bond wherein the molecule contains a protecting group that prevents the formation of a peptide bond;
coating a solid phase polymer layer on the surface of the solid support, wherein the solid phase polymer layer contains an electroactive compound that upon activation generates a second compound capable of catalyzing the removal of the protecting group;
activating the electroactive compound on at least a portion of the surface of the solid support by supplying a voltage to at least one electrode of the array of electrodes;
removing the solid phase polymer layer; and
coupling a second molecule capable of forming a peptide bond, wherein the molecule contains a protecting group that prevents the formation of a peptide bond, to a first molecule capable of forming a peptide bond that has been deprotected.

13. The method of claim 12 also including heating the solid support after supplying a voltage to at least one electrode of the array of electrodes.

14. The method of claim 12 wherein modulating the density of peptides to be formed on the support surface is accomplished by coupling a mixture of molecules capable of forming a peptide bond to the surface wherein the mixture contains molecules having a protecting group that prevents the formation of a peptide bond and molecules having a capping group that prevents the formation of a peptide bond.

15. The method according to claim 12 also including capping unreacted peptide bond-forming sites on the first molecule capable of forming a peptide bond after coupling the second molecule capable of forming a peptide bond.

16. The method according to claim 12 wherein attaching is accomplished through the formation of a peptide bond.

17. The method of claim 12 wherein a feature size of the array is less than 100 μm².

18. The method of claim 12 wherein the array contains 1,000 to 10,000 electrodes.

19. The method of claim 12 wherein the solid support is a silicon wafer.

20. The method of claim 12 wherein the surface of the solid support contains at least 500 electrodes that are individually addressable.

21. A method for making an array of polymers comprising on a solid support comprising,
providing an array of electrodes on a surface of the solid support;
attaching a first molecule capable of forming a phosphodiester bond wherein the molecule contains a protecting group that prevents the formation of a phosphodiester bond to the support surface;
coating a solid phase polymer layer on the surface of the solid support, wherein the solid phase polymer layer contains an electroactive compound that upon activation generates a second compound capable of catalyzing the removal of the protecting group;
activating the electroactive compound on at least a portion of the surface of the solid support by supplying a voltage to at least one electrode of the array of electrodes;
removing the solid phase polymer layer; and
coupling a second molecule capable of forming a phosphodiester bond, wherein the molecule contains a protecting group that prevents the formation of a phosphodiester bond, to a first molecule capable of forming a phosphodiester bond that has been deprotected.

22. The method according to claim 21 also including heating the solid support after supplying a voltage to at least one electrode of the array of electrodes.

23. The method of claim 21 wherein the solid support is a silicon wafer.

24. The method of claim 21 wherein a feature size of the array is less than 100 μm².

25. The method of claim 21 wherein the array contains 1,000 to 10,000 electrodes.

26. The method of claim 21 wherein the surface of the solid support contains at least 500 electrodes that are individually addressable.
